(19) United States
(12) Patent Application Publication
(10) Pub. No.: US 2023/0365615 A1
(43) Pub. Date: Nov. 16, 2023

(54) PROCESS OF OBTAINING CHOLESTEROL PRESENT IN FISH OIL

(71) Applicant: NATURMEGA S.A., Barranquilla (CO)

(72) Inventors: Wilson Martinez Gutierrez, Barranquilla (CO); Alfredo de Jesús Puche Simanca, Barranquilla (CO); Tatiana Lucia Yepes Bustillo, Barranquilla (CO); Alvaro Jose Garcia Padilla, Barranquilla (CO); Lorena Berrio Roa, Barranquilla (CO)

(21) Appl. No.: 18/029,269

(22) PCT Filed: Sep. 30, 2020
(86) PCT No.: PCT/IB2020/059184

§ 371 Date: Mar. 29, 2023

(51) Int. Cl. C07J 9/00

(52) U.S. Cl. CPC C07J 9/00; C11C 1/10

(57) ABSTRACT

An aspect of the present invention relates to a method for producing cholesterol from fish oil, which generally comprises the steps of transesterifying refined oil using a basic catalyst; obtaining a stream concentrated in cholesterol and another stream concentrated in omega-3 fatty acids EPA and DHA by means of molecular distillation; and saponifying the cholesterol-rich stream using bases such as potassium hydroxide, calcium hydroxide, sodium hydroxide or calcium chloride. The mixture is subsequently saponified over a range of time and temperature, with agitation and constant reflux. Multiple extractions are carried out using solvents such as toluene, ethylene dichloride, methylene chloride, diethyl ether, petroleum ether, acetone, benzene or a hexane, and centrifugations are performed to obtain the unsaponifiable matter. This last stream is crystallised using acetone, benzene, toluene, diethyl ether, petroleum ether or a hexane to obtain cholesterol with at least 95% purity. In addition, the soap obtained from saponification is transformed into a form of ethyl ester by applying breakdown reactions and acidic esterification.

### FIELD OF THE INVENTION

The present invention relates to a process for separating the cholesterol present in fish oil. Particularly, it comprises a process for separating the cholesterol present in fish oil by methods of transesterification of the refined oil, molecular distillation, saponification of a heavy fraction, and extraction and purification of cholesterol through crystallization. Based on the cholesterol separation process of the invention, a concentration of contaminants which are persistent and bioaccumulate is not generated. Therefore, the present invention provides a process for obtaining pharmaceutical grade cholesterol from fish oil having at least 90% cholesterol and simultaneously producing a high quality residual or processed fish oil suitable for human or animal consumption or for processing of EPA and DHA concentrates among others at the pharmaceutical level.

Cholesterol is a sterol (or modified steroid), lipid molecule and is biosynthesized by animal cells as it is an essential structural component of animal cell membranes, which is required to maintain both membrane structural integrity and fluidity. This substance can be found in various sources, such as egg yolk, spinal cord, bones, brain tissue of cows, goat, pig, poultry, fish organs and shrimp exoskeleton and it is even possible to find it in vegetable oils in low concentrations. However, the most important source of cholesterol is lanolin, obtained from sheep's wool. These sources generally contain cholesterol in its free form, as well as in the esterified form (cholesterol molecule bound to a fatty acid).

Cholesterol is an important pharmaceutical intermediate required for the biosynthesis of steroid hormones, bile acids, and vitamin D3. There is currently an increasing demand for pharmaceutical grade cholesterol having a cholesterol content of 90% or more for the production of vitamins D3, vitamin D3 derivatives, hormones and emulsions in cosmetics. Among other uses is the synthesis of anti-inflammatory steroid drugs, production of creams for skin lubrication and cosmetics. Other cholesterols with a much lower purity are used for feeding crustaceans and aquaculture.

Cholesterol can be found in egg yolks, organ meats, shrimp, calamari, beef, pork, poultry, fish, wool grease, full-fat dairy products, butter, hard margarines, lard, coconut oil, butter (clarified butter), vegetable shortening and palm oil. These sources generally contain cholesterol in its free form, as well as in the esterified form. The most important source of cholesterol is lanolin, which is obtained from sheep's wool. However, since the fatty acid composition in wool fat is complex in nature due to the presence of free acids and alcohols that can form variable esters, cholesterol extraction is cumbersome and expensive.

Crude fish oils mainly contain triglycerides that make up about 90% of the composition. The other components of the composition comprise partial glycerides (ie, mono- or diglycerides), free fatty acids, phospholipids, and a group of chemicals as the non-saponifiable fraction that includes cholesterol-sterol, glyceryl ethers, fatty alcohols, vitamins, and oxidized pigments. The content of the unsaponifiable fraction varies with seasonal and feeding conditions and varies in the range of 2-8%.

Fish oil residues from the fish refining industry contain useful products such as cholesterol, proteins, and enzymes, among other fatty acids. Due to the importance of cholesterol as a precursor in the manufacture of vitamin D3, fish oil and fish oil residues (after removal of polyunsaturated fatty acids) that form the waste stream in the fish oil industry are now considered as an alternative source of cholesterol.

Due to the importance of cholesterol as an intermediate in the production of vitamin D and its derivatives, there is always a need to provide alternative and improved isolation methods for cholesterol production.

### BACKGROUND OF THE INVENTION

Currently, cholesterol on an industrial scale is produced primarily from wool wax alcohols, ie, the unsaponifiable fraction of wool fat, which contains from about 25% to about 32% cholesterol. The most common processes for producing cholesterol involve the formation of an insoluble addition product by reacting cholesterol with a metal salt, followed by decomposition and recovery of the cholesterol. Such processes can meet the purity requirements for cholesterol applications in the pharmaceutical industry. In this sense, the state of the art has presented a variety of disadvantages. In particular, some prior art examples include States U.S. Pat. No. 10,190,074 by Meza et al., which discloses a process for producing a cholesterol concentrate that includes the steps of distilling a fish oil that has no more than 2% free fatty acids in a mixture with an auxiliary fluid in a vacuum distillation column to obtain a first distillate and a first residue; and distilling the first distillate in a vacuum distillation column to obtain a second distillate and a second residue, wherein the second residue includes the cholesterol concentrate.

These prior art processes of using high temperatures during molecular distillation generates degradation of the omega-3 fatty acids such as EPA (eicosapentanoic acid) and DHA (docosahexanoic acid), so that the content of trans fatty acids is increased and further promotes the polymerization of unsaturated fatty acids.

The same happens with the teachins of Great Britain patent GB489623A that uses a material that contains a compound that has a cholesteric nucleus, such as sterols, saponins and bile acids, which are subjected to a short path high vacuum distillation to separate the substance thereof, and then the compound is purified. Specified raw materials include animal oils and waxes, such as whale oil, fecal fats and oils, and china wax; vegetable oils, such as soybean oil, cottonseed oil, wheat germ oil, and bean oil; and fungal growths, such as ergot, each of which are sources of zoosterols, phytosterols, and mycosterols, respectively. In the same way, in Japanese patent JPS62145099A by Hata et al., which discloses a fish oil, preferably subjected to degumming treatment, obtained from a fish such as sardines, mackerel, saury fish, etc., it is subjected to molecular distillation using, for example, in an apparatus such as a falling film type, or centrifugal type.

Furthermore, the above prior art reveals temperatures that generate large concentrations of pollutants such as polychlorinated biphenyls (PCB's), polyaromatic hydrocarbons (PAH), heavy metals, pesticides and degradation products of the aforementioned compounds, which are persistent and bioaccumulative.

However, starting from a raw material other than fish oil, such as wool fat/lanolin, does not allow the recovery of fatty acids in the form of saponified soap during cholesterol esterification. The obtained soap is split to obtain free fatty acids and then esterified by means of an acid catalyst to obtain fatty acids in the form of ethyl ester. Starting from the resulting ethyl ester, it is possible to concentrate omega-3 by means of molecular distillation.

GB526951 describes a process for the extraction of cholesterol from animal tissues such as the brain, spinal cord, etc. by saponification and extraction with a water immiscible solvent. These known processes generate large amounts of liquid industrial waste, the management and/or disposal of which significantly increases production costs.

Given the problems of the prior art, the inventors have discovered that cholesterol can be extracted with high yields and good quality from fish oil by ultrasonic transesterification, molecular distillation, saponification, solvent extraction and cholesterol purification methods through crystallization.

### OBJECTS OF THE INVENTION

Therefore, a first object of the present invention is to avoid the drawbacks of the prior art. Particularly, the main object of the present invention is to create a process for separating the cholesterol present from fish oil, which process does not generate any amounts of contaminants. According to the present invention, the main object of the invention is to provide beneficial processes wherein cholesterol purity of at least 95% is obtained, wherein such processes can be conducted on an industrial scale capable of satisfying the market demand for pharmaceutical grade cholesterol, as the raw material. It is also commonly easy to obtain and provide an alternative for use of a co-product used to feed crustaceans, which production cost is low and the solvents used are recovered and reused in the production process.

Another no less important object is the separation of cholesterol present in the heavy fraction from fish oil by methods of saponification and extraction and purification of cholesterol through crystallization.

Finally, another object of the invention also resides in the relationship with molecular distillation or short-path distillation. This is an operation technique for liquid-liquid separation done continuously under vacuum pressure. Considering a high vacuum pressure, the distance between the heating surface and the surface of the internal condenser of the evaporator is less than or equal to the mean free path of the molecules during the separation. In this process, a thin film is formed on the hot surface and the most volatile compounds evaporate, condensing when they come into contact with the internal condenser of the evaporator. The present invention meets these needs and provides other related advantages, including obtaining pharmaceutical grade cholesterol from fish oil with a purity of at least 95%.

The novel features which are considered to be the basis of the invention are set forth in particular in the appended claims and the additional advantages thereof, will be better understood from the following detailed description of the preferred embodiments.

### DETAILED DESCRIPTION OF THE INVENTION

The present invention relates to a process for separating cholesterol present in fish oil by methods of transesterification, molecular distillation, saponification, extraction and purification of cholesterol through crystallization.

In one embodiment, the invention then refers to the process based on the separation of cholesterol present in fish oil by the method of transesterification, molecular distillation, saponification, solvent extraction and purification through crystallization and obtaining cholesterol in crystalline form.

In one aspect, the present invention refers to a process for producing cholesterol from fish oil, which generally comprises the steps of esterifying the refined oil with an acid value of less than 2 mg KOH/g using alcohol under the action of a basic catalyst with the aim of converting the glycerides present in the oil to the ethyl ester form or other esters.

The alcohol used for the present invention may be selected from the group consisting of methanol, propanol, butanol, ethanol, glycerin or propylene glycol, and the catalyst may be selected from the group consisting of sodium hydroxide, potassium hydroxide, sodium ethoxide or sodium methoxide.

Next, the process of the invention comprises concentrating the cholesterol present in the ethyl ester obtained by using a series of short-path evaporators, and then obtaining in turn another stream having a high concentration of EPA and DHA.

Subsequently, dissolve the concentrated cholesterol in a base in a determined amount in water with the addition of an alcohol. The base used in the present invention may comprise, without being limited to, potassium hydroxide, calcium hydroxide, sodium hydroxide, or calcium chloride.

Subsequently the mixture is saponified for a period of time and temperature with constant stirring and reflux. Subsequently, a solvent is added with stirring and alcohol is added to promote correct separation. The above step is followed by a centrifugation process to separate the soap from the extracted phase.

In other aspects of the invention, it is important that a new extraction with a second solvent must be carried out with stirring and addition of alcohol to increase the separation of the unsaponifiable matter contained in the solvent. The solvent is then evaporated using a thin film evaporator to obtain dry unsaponifiable matter. Subsequently, a wash is carried out with a solution of sodium hydroxide or calcium chloride and it is then centrifuged to eliminate the washing water and then a solvent such as acetone, benzene or toluene is added and a first crystallization is carried out. A second solvent selected from the group consisting of hexane, diethyl ether, petroleum ether, acetone and benzene may also be used. Centrifugation and filtration are then carried out and a second crystallization of the previously obtained crystals is carried out with an organic solvent such as diethyl ether or petroleum ether or hexane.

Finally, a new centrifugation and filtration is carried out to obtain cholesterol crystals with a purity greater than 95%.

On the other hand, the soap obtained from the last centrifugation is broken down with an acid in the presence of water at a given temperature and reaction time with agitation. Then, it is cooled and the water is separated from the free fatty acids using a centrifuge. Acids suitable for this step include sulfuric, hydrochloric, citric, acetic or phosphoric acid.

The free fatty acids are then pumped to an ultrasonic reactor, where they are esterified with an alcohol and an acid catalyst at a given temperature and reaction time with constant stirring and reflux to obtain fatty acids in the form of the ethyl ester. Said acid catalyst can be selected without being limited from the group consisting of citric acid, acetic acid, phosphoric acid, hydrochloric acid, p-toluenesulfonic acid or sulfuric acid.

Therefore, the invention initially provides a transesterification and molecular distillation process that includes a first stage for obtaining fatty acids in the form of the ethyl esters and which comprises:
i. A first step of transesterifying refined fish oil with an acid value of less than 2 mg KOH/g by reacting with methanol, propanol, butanol or ethanol in a ratio between 1:5 and 1:2 w/w with respect to the oil refined, preferably 1:3 w/w with the addition of a basic catalyst such as sodium hydroxide, potassium hydroxide, sodium ethoxide or sodium methoxide in a concentration between 0.75 and 1.5% w/w with respect to the oil, said reaction conducted in four ultrasound reactors arranged in series for a time between 20 and 60 minutes, at a pressure between 1 and 3 bar, at a temperature between 60° C. and 90° C. and a constant reflux of alcohol;
ii. A second step to recover the alcohol not consumed in the reaction by flash evaporation in an evaporator that operates between 0.2 and 1 bar of pressure and a temperature between 50 and 80° C., where the alcohol recovered in this step is reusable in the first step;
iii. A third step to separate the mixture of ethyl ester and glycerin obtained from the transesterification by means of a disc centrifuge that operates between 3500 and 6000 RPM, and the reaction reaches a conversion of glycerides to ethyl ester of at least 95%;
iv. A fourth step of concentrating the cholesterol present in the oil in the form of ethyl ester through molecular distillation by obtaining a heavy fraction, using short-path distillation units arranged in series, where the residue from one evaporator is fed to the next distillation unit and the distillate it is then returned to the previous still, where the operating conditions of the stills comprise vacuum pressures from 0.1 to 10 Pa, temperatures between 120 and 165° C. and heat transfer areas between 2 and 5 m² and where in this step a stream with a high cholesterol content and another ethyl ester stream with a high content of EPA and DHA fatty acids are obtained.

Regarding the fourth step, it must be understood that according to the process of the invention, two streams are obtained: (a) one with a high cholesterol content and (b) another with a high content of EPA and DHA.

The invention continues with a second stage of saponification and extraction that includes:
v. A fifth step to obtain a soap using a base selected from the group consisting of calcium hydroxide, sodium hydroxide, potassium hydroxide or calcium chloride in water with a concentration between 10% and 30% w/w, and also adding an alcohol such as ethanol, methanol, glycerin or propylene glycol with a ratio between 2 to 5% w/w, preferably 3% w/w with respect to the base solution in a high-efficiency mixer, where it is mixed with the concentrated fraction of cholesterol in weight ratio between 1:0.9; 1:1.1; 1:1.2, preferably 1:1 with respect to the stream coming from molecular distillation and where the mixture enters a reactor where it is saponified in a time between 1 and 4 hours, preferably 1 to 2 hours, at a temperature between 60° C. and 85° C. with stirring at speeds from 500 to 1050 RPM with constant reflux;

vi. A sixth extraction step where the soap from the fifth step enters an extractor column, in which a solvent selected from the group consisting of toluene, ethylene dichloride, methylene chloride, preferably ethylene dichloride, is added at a 4:1 ratio, or 1:1, preferably 3:1 with respect to the added soap, with constant stirring at a speed between 200 RPM and 750 RPM, and at a temperature between 30-50° C.;
vii. An optional seventh step to favor the subsequent separation of phases which step includes the addition of ethanol, methanol, propanol or butanol in a concentration of 0 to 5% by weight with respect to the mixture of the extraction solvent and the soap;
viii. An eighth step that comprises centrifuging the extraction mixture to separate the soap and the solvent at a speed range between 500 and 3500 rpm, preferably 1000 rpm;
ix. A ninth step which comprises a new extraction of the centrifuged soap from the eighth step in an extractor column, in which diethyl ether, petroleum ether, acetone, benzene or hexane are added, at a 4:1 to 1:1 ratio, with respect to the fed soap, with constant agitation and at a temperature between 30 to 55° C. and accompanied by the favoring of phase separation of the seventh step, where it also includes the centrifugation of the extraction mixture based on the eighth step.
x. A tenth step that includes performing the steps from the sixth to ninth steps between 1 to 3 more times;
xi. An eleventh step where the solvent from the dispersing phases of each one of the centrifugations contemplated in the eighth, ninth and tenth steps is evaporated and recovered by means of a battery of evaporators arranged in series, which evaporators operate under pressures from 10 to 100 Pa and at temperatures between 30 to 120° C., where after evaporation, the unsaponifiable matter (MI) that was dissolved in the solvent is recovered;
xii. A twelfth step where the soap obtained from the last centrifugation of the tenth step is arranged for a breakdown with an acid selected from the group consisting of sulfuric acid, hydrochloric acid, acetic acid, phosphoric acid or citric acid in aqueous solution at 50 to 70% w/w in an ultrasonic reactor, where the ratio of soap to acid solution is between 3:1 to 1:1, preferably 2:1 and a temperature between 70° C. and 90° C. for 1 to 3 hours with constant stirring. Then, it is cooled and the water is separated from the free fatty acids formed using a centrifuge that operates in a range between 450 and 850 RPM;
xiii. A thirteenth step that contemplates the esterification of the free fatty acids with ultrasound using ethanol, methanol or propanol in relation to the free fatty acid by weight between 1:5 to 1:3, preferably 1:4 and an acid catalyst such as sulfuric acid, p-toluenesulfonic acid, citric acid, acetic acid, phosphoric acid or hydrochloric acid dissolved in alcohol at a concentration of 3 to 8% w/w for a time of one to three hours at a temperature between 65° C. and 85° C. with stirring and constant reflux. Finally, a stream of fatty acids in the form of ethyl ester is obtained.

After obtaining cholesterol, it is necessary to purify it. For this purpose, the process additionally includes a third stage that includes:
xiv. A fourteenth step where the unsaponifiable matter is washed with an aqueous solution of sodium hydroxide or calcium chloride at 5-10%. The washing is carried out in a proportion of 1:2 and 1:4 by weight with respect to the desolventized unsaponifiable matter in a temperature range between 40 and 60° C.;
xv. A fifteenth step that includes centrifugation of the mixture obtained after washing to separate the unsaponifiable matter that was treated and the washing water, wherein this step is executed in a range of speeds between 500 and 3500 RPM, preferably 2000 RPM;
xvi. A sixteenth step where an organic solvent selected from acetone, benzene or toluene is added to the unsaponifiable material from the fifteenth step at a ratio of 15:1 to 5:1, heated to 30° C. and stirred until dissolved. It is then crystallized at a temperature between −10° C. and 5° C., then maintained under isothermal conditions at 5° C. for 8 hours, and then cooled to −10° C. for 10 hours.
xvii. A seventeenth step where it is centrifuged and filtered using a basket centrifuge to obtain cholesterol crystals with a purity greater than 70%.
xviii. An eighteenth step where another organic solvent selected from diethyl ether, petroleum ether or hexane is added to 70% purity cholesterol from the seventeenth step at a ratio of 15:1 to 5:11, heated to 30° C. and it is stirred until dissolution, where it then crystallizes at a temperature between −40° C. and 0° C., maintaining under isothermal conditions at −5° C. for 10 hours, and then cooled to −40° C. for 8 hours;
xix. A nineteenth step where it is centrifuged and filtered using a basket centrifuge to obtain cholesterol crystals with a purity greater than 95%.

For the present invention, the set of evaporators makes it possible to recover and purify the solvent used to reuse it in the extraction process. The product of this stage are crystals of unsaponifiable matter.

Based on the cholesterol separation process of the invention, a concentration of contaminants which are persistent and bioaccumulate is not generated.

Similarly, the invention can be reflected from the examples described below.

### Example 1

1. 2000 kg of fish oil with an acid value of 1.37 mg KOH/g were transesterified with 600 kg of ethanol and 15 kg of potassium hydroxide in four ultrasonic reactors arranged in series within a period of 50 minutes, at a pressure of 3 bars, at a temperature of 65° C. and a constant reflux of alcohol;
2. The ethanol that was not consumed by the reaction was recovered in an evaporator operating at a pressure of 0.5 bar and 60° C.
3. The ethyl ester and glycerin mixture was separated using a disc centrifuge with a spin speed of 4500 RPM. Once the glycerin was removed, the analysis of partial glycerides and ethyl ester confirmed that the ethyl ester concentration was 95.9%;
4. Eight short-path stills were arranged in series, where the residue from one still is fed to the next still and the distillate is returned to the previous still, at temperature ranges between 125 and 160° C. and vacuum pressures from 0.1 to 7 Pa. At the end of this stage, two streams were obtained: a stream with a high cholesterol content and another stream with a high content of EPA and DHA fatty acids.
5. 100 kg of the stream with high concentration of cholesterol was obtained, which was then saponified using 100 kg of an aqueous solution of sodium hydroxide at 15% w/w, and the addition of 2 kg of propylene glycol. The reaction time was two hours at a temperature of 75° C., and stirring at 750 RPM and with constant reflux;
6. The obtained soap then enters an extractor column in which a liquid-liquid extraction is carried out using toluene in a ratio of 3 to 1 by weight with respect to the soap that is fed into the system. The extraction is carried out with constant stirring at 400 RPM and a temperature of 40 C.
7. 1% ethanol w/w is added with respect to the soap and a solvent mixture is obtained for subsequent separation.
8. The extraction mixture is centrifuged for the separation of soap and solvent using a disc centrifuge at 1000 RPM;
9. A new extraction of the previously extracted soap was carried out in another extracting column using acetone in a 3:1 ratio with respect to the soap being fed, with constant stirring at 350 RPM and at a temperature of 45° C. In addition, it was then centrifuged at 1000 RPM to separate the solvent from the soap.
10. Additionally, three extractions and three more centrifugations were carried out as previously described.
11. The solvent from the dispersing phases of each of the centrifugations conducted in the previous steps was evaporated and recovered by means of a battery of evaporators arranged in series, which are operated under pressures from 10 to 80 Pa and temperatures between 40 to 90° C., wherein after evaporation, the unsaponifiable matter (MI) that was dissolved in the solvent was obtained.
12. The soap obtained from the last centrifugation conducted in the tenth step, that is, the most depleted soap, was arranged for treatment with an acid selected from citric acid in an aqueous solution at 67% w/w in an ultrasonic reactor, where the ratio of soap with respect to the acid solution was 2:1 by weight and at a temperature of 75° C. for 2 hours with constant stirring. Then, the resulting reaction mixture was cooled using water from a cooling tower and the water was separated from the free fatty acids formed using a centrifuge that operated at 650 RPM.
13. The esterification of the free fatty acids was carried out in an ultrasonic reactor using ethanol, feeding 1 part of activated ethanol for every 4 parts by weight of free fatty acids. Ethanol activation was performed by dissolving 7.5% p-toluenesulfonic acid in alcohol. The reaction was carried out for 1.5 hour at a temperature of 70° C. with stirring and a constant reflux of ethanol. Finally, a stream of fatty acids was obtained in the form of ethyl ester.
14. Additionally, a wash was carried out on the previously obtained unsaponifiable matter, using a 10% aqueous solution of calcium chloride. The washing was carried out in a ratio of 1:3 by weight with respect to the desolventized unsaponifiable matter, at a washing temperature of 45° C.
15. The separation of the washing solution was carried out by centrifugation, which stage was carried out at 2000 RPM.
16. 12 parts of acetone were then added for each part by weight of unsaponifiable matter. It was then heated to 30° C. and stirred until dissolved. Then it was crystallized between 5° C. and −10° C., maintaining isothermal conditions at 5° C. for 8 hours, then cooling to −10° C. and further maintaining isothermal conditions at this temperature for 10 hours.
17. The resulting product was centrifuged and filtered using a basket centrifuge to obtain cholesterol crystals with a purity of 74.5%.
18. Then 12 parts of petroleum ether were added to each part of cholesterol with a purity greater than 70%, and then the mixture was heated to 30° C. and stirred until dissolution, where it then crystallized at a temperature between −40° C. and 0° C., maintaining isothermal conditions at −5° C. for 10 hours, then cooling to −40° C. and then further maintaining isothermal conditions at this temperature for 8 hours.
19. Finally, the separation of the crystals obtained from the solvent is carried out using a basket centrifuge in to obtain cholesterol crystals with a purity of 96.5%.

### Example 2

1. 2000 kg of fish oil with an acid value of 1.37 mg KOH/g were transesterified with 600 kg of ethanol and 15 kg of sodium ethoxide in four ultrasonic reactors arranged in series within a period of 50 minutes, at a pressure of 3 bars, at a temperature of 65° C. and a constant reflux of alcohol.
2. The ethanol that was not consumed by the reaction was recovered in an evaporator operating at a pressure of 0.5 bar and 60° C.
3. The ethyl ester and glycerin mixture was separated using a disc centrifuge with a spin speed of 4500 RPM. Once the glycerin was removed, the analysis of partial glycerides and ethyl ester confirmed that the ethyl ester concentration was 96.9%.
4. Eight short-path stills were arranged in series, where the residue from one still is fed to the next still and the distillate is returned to the previous still, at temperature ranges between 125 and 160° C. and vacuum pressures from 0.1 to 7 Pa. At the end of this stage, two streams were obtained: a stream with a high cholesterol content and another stream with a high content of EPA and DHA fatty acids.
5. 100 kg of the stream with a high concentration of cholesterol was obtained, which was then saponified using 100 kg of an aqueous solution of potassium hydroxide at 15% w/w. The reaction time was two hours at a temperature of 75° C., stirring at 750 RPM and with constant reflux. 6. The obtained soap then enters an extractor column in which a liquid-liquid extraction is carried out using ethylene dichloride in a ratio of 3 to 1 by weight with respect to the soap being fed. The extraction is carried out with constant stirring at 400 RPM and a temperature of 40° C.
7. The extraction mixture is centrifuged for the separation of soap and solvent in a disc centrifuge at 1000 RPM;
8. A new extraction of the previously extracted soap was then carried out in another extractor column using petroleum ether in a 3:1 ratio with respect to the soap being fed, using constant stirring at 350 RPM, accompanied by the addition of 1% ethanol for favoring the subsequent separation of the phases. In addition, it was then centrifuged at 1000 RPM to separate the solvent from the soap.
9. Three extractions and three more centrifugations were further carried out as previously described.
10. The solvent of the dispersing phases from each of the centrifugations conducted in the previous steps was evaporated and recovered by means of a battery of evaporators arranged in series, which operated under pressures from 10 to 80 Pa and at temperatures between 40 to 90° C., wherein after evaporation, the unsaponifiable matter (MI) that was dissolved in the solvent was obtained.
11. The soap obtained from the last centrifugation of the tenth step, that is, the most spent soap, was arranged for acidification with an acid selected from citric acid in an aqueous solution at 67% w/w in an ultrasonic reactor, where the ratio of soap to with respect to the acid solution was 2:1 by weight. The process is conducted at a temperature of 75° C. for 2 hours with constant stirring. Then, it was cooled using water from a tower and the water was separated from the free fatty acids that were formed using a centrifuge that operated at 650 RPM.
12. The esterification of the free fatty acids was carried out in an ultrasonic reactor using ethanol, feeding 1 part of activated ethanol for every 4 parts by weight of free fatty acids. The ethanol activation was carried out by dissolving 7.5% sulfuric acid in the alcohol. The reaction was carried out for 1.5 hour at a temperature of 70° C. with stirring and constant reflux of ethanol. Finally, a stream of fatty acids was obtained in the form of ethyl ester.
13. Additionally, a further washing step was carried out on the previously obtained unsaponifiable matter, using a 10% aqueous sodium hydroxide solution. The washing was carried out in a ratio of 1:3 by weight with respect to the desolventized unsaponifiable matter at a washing temperature of 45° C.
14. The separation of the washing solution was carried out by centrifugation, which centrifugation was carried out at 2000 RPM.
15. 12 parts of toluene were then added for each part by weight of unsaponifiable matter, and then it was heated to 30° C. and stirred until dissolved. Then it was crystallized between 5° C. and −10° C., under isothermal conditions at 5° C. for 8 hours, then cooled to −10° C. under isothermal conditions at this temperature for 10 hours.
16. The resulting product of step 15 was then centrifuged and filtered using a basket centrifuge to obtain cholesterol crystals with a purity of 66.4%.
17. 12 parts of petroleum ether were then added to each part of cholesterol with a purity greater than 70%, and the mixture was heated to 30° C. and stirred until dissolution, wherein it then crystallized at a temperature between −40° C. and 0 C, and then maintaining isothermal conditions at −5 C for 10 hours, and then cooling to −40° C. under isothermal conditions at this temperature for 8 hours.
18. Finally, the separation of the crystals obtained from the solvent is carried out using a basket centrifuge in order to obtain cholesterol crystals having a purity of 92.1%.

### Example 3

1. 2000 kg of fish oil with an acid value of 1.37 mg KOH/g were transesterified with 600 kg of ethanol and 15 kg of sodium ethoxide in four ultrasonic reactors arranged in series within a period of 50 minutes, at a pressure of 3 bar, at a temperature of 65° C. and a constant reflux of alcohol.
2. The ethanol that was not consumed by the reaction was recovered in an evaporator that operates at a pressure of 0.5 bar and 60° C.
3. The ethyl ester and glycerin mixture was separated using a disc centrifuge using a spin speed of 4500 RPM. Once the glycerin was removed, the analysis of partial glycerides and ethyl ester confirmed that the ethyl ester concentration was 96.3%.
4. Eight short-path stills were then arranged in series, where the residue from one still is fed to the next still and the distillate is returned to the previous still. The stills are operated at temperature ranges between 125 and 160° C. and at vacuum pressures from 0.1 to 7 Pa. At the end of this stage, two streams were obtained: a stream with a high cholesterol content and another stream with a high content of EPA and DHA fatty acids.
5. 100 kg of the stream with a high concentration of cholesterol which was obtained, was saponified using 100 kg of an aqueous solution of potassium hydroxide at 15% w/w. The reaction time was two hours at a temperature of 75° C., stirring at 750 RPM and with constant reflux.
6. The obtained soap then enters an extractor column in which a liquid-liquid extraction is carried out using toluene in a ratio of 3 to 1 by weight with respect to the soap being fed. The extraction is carried out using constant stirring at 400 RPM and a temperature of 40° C.
7. The extraction mixture is then centrifuged for the separation of soap and solvent using a disc centrifuge at 1000 RPM.
8. A new extraction of the previously extracted soap was carried out in another extracting column using acetone in a 3:1 ratio with respect to the soap being fed, with constant stirring at 350 RPM, accompanied by the addition of 1% ethanol to promote subsequent phase separation. In addition, it was then centrifuged at 1000 RPM to separate the solvent from the soap.
9. Three extractions and three more centrifugations were carried out as previously described above.
10. The solvent of the dispersing phases of each of the centrifugations conducted in the previous steps was evaporated and recovered by means of a battery of evaporators arranged in series, which evaporators operated under pressures from 10 to 80 Pa and temperatures between 40 to 90° C. After evaporation, the unsaponifiable matter (MI) that was dissolved in the solvent was obtained.
11. The soap obtained from the last centrifugation of the tenth step, that is, the most spent soap, was arranged for acidification with an acid selected from citric acid in an aqueous solution at 67% w/w in an ultrasonic reactor, where the ratio of soap to with respect to the acid solution was 2:1 by weight and a temperature of 75° C. for 2 hours with constant stirring. Then, it was cooled using water from a cooling tower and the water was separated from the free fatty acids formed using a centrifuge that operated at 650 RPM.
12. The esterification of the free fatty acids was carried out in an ultrasonic reactor using ethanol, feeding 1 part of activated ethanol for every 4 parts by weight of free fatty acids. The ethanol activation was carried out by dissolving 7.5% sulfuric acid in the alcohol. The reaction was carried out for 1.5 hour at a temperature of 70° C. with stirring and constant reflux of ethanol. Finally, a stream of fatty acids was obtained in the form of the ethyl ester.
13. Additionally, a wash was carried out on the previously obtained unsaponifiable matter, using a 10% aqueous sodium hydroxide solution. The washing was carried out in a ratio of 1:3 by weight with respect to the desolventized unsaponifiable matter, and the washing temperature was 45° C.
14. The separation of the washing solution was carried out by centrifugation and this stage was carried out at 2000 RPM.
15. 12 parts of acetone were added for each part of unsaponifiable matter, then it was heated to 30° C. and stirred until dissolved. Then it was crystallized between 5° C. and −10° C., under isothermal conditions at 5° C. for 8 hours, then cooled to −10° C. and maintaining isothermal conditions at this temperature for 10 hours.
16. It was then centrifuged and filtered using a basket centrifuge to obtain cholesterol crystals with a purity of 63.5%.
17. 12 parts of petroleum ether were added to each part of cholesterol with a purity greater than 70%, and the mixture was heated to 30° C. and stirred until dissolution, where it then crystallized at a temperature between −40° C. and 0° C., and further maintaining it under isothermal conditions at −5° C. for 10 hours, then cooling to −40° C. and then maintaining isothermal conditions at this temperature for 8 hours.
18. Finally, the separation of the crystals obtained from the solvent is carried out using a basket centrifuge in order to obtain cholesterol crystals with a purity of 94.1%.

### Example 4

1. 2000 kg of fish oil with an acid value of 1.37 mg KOH/g were transesterified with 600 kg of ethanol and 15 kg of potassium hydroxide in four ultrasonic reactors arranged in series within a period of 50 minutes, at a pressure of 3 bars, at a temperature of 65° C. and constant reflux of alcohol;
2. The ethanol that was not consumed by the reaction was recovered in an evaporator that operates at a pressure of 0.5 bar and 60° C.
3. The ethyl ester and glycerin mixture was separated using a disc centrifuge with a spin speed of 4500 RPM. Once the glycerin was removed, the analysis of partial glycerides and ethyl ester confirmed that the ethyl ester concentration was 94.9%.
4. Eight short-path stills were arranged in series, where the residue from one still is fed to the next still and the distillate is returned to the previous still, at temperature ranging between 125 and 160° C. and vacuum pressures from 0.1 to 7 Pa. At the end of this stage, two streams were obtained: a stream with a high cholesterol content and another stream with a high content of EPA and DHA fatty acids.
5. 100 kg of the stream with high concentration of cholesterol were obtained, which was saponified using 100 kg of an aqueous solution of potassium hydroxide at 15% w/w, and the addition of 2 kg of ethanol. The reaction time was two hours at a temperature of 75° C., with stirring at 750 RPM and with constant reflux.
6. The soap obtained then enters an extractor column in which a liquid-liquid extraction is carried out using ethylene dichloride in a ratio of 3 to 1 by weight with respect to the soap being fed. The extraction is carried out with constant stirring at 400 RPM and a temperature of 40° C.
7. The extraction mixture is centrifuged for the separation of soap and solvent in a disc centrifuge at 1000 RPM.
8. A new extraction of the previously extracted soap was carried out in another extraction column using hexane in a 3:1 ratio with respect to the soap being fed, with constant stirring at 350 RPM and at a temperature of 45° C. In addition, it was centrifuged at 1000 RPM to separate the solvent from the soap.
9. Three extractions and three more centrifugations were carried out as previously described above.
10. The solvent from the dispersing phases of each of the centrifugations contemplated in the previous steps was evaporated and recovered by means of a battery of evaporators arranged in series, which operated under pressures from 10 to 80 Pa and at temperatures between 40 to 90° C., where after evaporation, the unsaponifiable matter (MI) that was dissolved in the solvent was obtained.
11. The soap obtained from the last centrifugation of the tenth step, that is, the most spent soap, was arranged for acidification with an acid selected from citric acid in an aqueous solution at 67% w/w in an ultrasonic reactor, where the ratio of soap to the acid solution was 2:1 by weight and at temperature of 75° C. for 2 hours with constant stirring. Then, it was cooled using water from a tower and the water was separated from the free fatty acids formed using a centrifuge that operated at 650 RPM.
12. The esterification of free fatty acids was carried out in an ultrasonic reactor using ethanol, by feeding 1 part of activated ethanol for every 4 parts by weight of free fatty acids. Ethanol activation was performed by dissolving 7.5% p-toluenesulfonic acid in alcohol. The reaction was carried out for 1.5 hour at a temperature of 70° C. with stirring and a constant reflux of ethanol. Finally, a stream of fatty acids was obtained in the form of the ethyl ester.
13. Additionally, a wash was carried out on the previously obtained unsaponifiable matter, using a 10% aqueous solution of calcium chloride. The washing was carried out in a ratio of 1:3 by weight with respect to the desolventized unsaponifiable matter, at a washing temperature of 45° C.;
14. The separation of the washing solution was carried out by centrifugation, and this stage was carried out at 2000 RPM.
15. 12 parts of hexane were added for each part of unsaponifiable matter, then heated to 30° C. and stirred until dissolved. Then it was crystallized between 5° C. and −10° C., then maintained under isothermal conditions at 5° C. for 8 hours, then cooling to −10° C. and further maintaining it under isothermal conditions at this temperature for 10 hours.
16. It was then centrifuged and filtered using a basket centrifuge to obtain cholesterol crystals with a purity of 74.5%.
17. 12 parts of hexane were then added to each part of cholesterol with a purity greater than 70%, and the mixture was heated to 30° C. and stirred until dissolved, where it then crystallized at a temperature between −40° C. and 0° C., and then maintaining isothermal conditions at −5° C. for 10 hours, then cooling to −40° C. and further maintaining isothermal conditions at this temperature for 8 hours.
18. Finally, the separation of the crystals obtained from the solvent is carried out using a basket centrifuge in order to obtain cholesterol crystals with a purity of 90.2%.

### Example 5

1. 2000 kg of fish oil with an acid value of 1.37 mg KOH/g were transesterified with 600 kg of ethanol and 15 kg of sodium ethoxide in four ultrasonic reactors arranged in series within a period of 50 minutes, at a pressure of 3 bar, with a temperature of 65° C. and a constant reflux of alcohol.
2. The ethanol that was not consumed by the reaction was recovered in an evaporator that operates at a pressure of 0.5 bar and 60° C.
3. The ethyl ester and glycerin mixture was separated using a disc centrifuge with a spin speed of 4500 RPM. Once the glycerin was removed, the analysis of partial glycerides and ethyl ester confirmed that the ethyl ester concentration was 96.3%.
4. Eight short-path stills were arranged in series, where the residue from one still is fed to the next still and the distillate is returned to the previous still, at temperature ranging between 125 and 160° C. and vacuum pressures from 0.1 to 7 Pa. At the end of this stage, two streams were obtained: one stream with a high cholesterol content and another stream with a high content of EPA and DHA fatty acids.
5. 100 kg of the stream with a high concentration of cholesterol was obtained, which was then saponified using 100 kg of an aqueous solution of sodium hydroxide at 15% w/w. The reaction time was two hours at a temperature of 75° C., with stirring at 750 RPM and with constant reflux.
6. The soap obtained then enters an extractor column in which a liquid-liquid extraction is carried out using toluene in a ratio of 2 to 1 by weight with respect to the soap being fed. The extraction is carried out with constant stirring at 400 RPM and at a temperature of 40° C.
7. The extraction mixture is centrifuged for the separation of soap and solvent in a disc centrifuge at 1000 RPM.
8. A new extraction of the previously extracted soap was carried out in another extracting column using acetone in a ratio of 2 to 1 with respect to the soap being fed, with constant stirring at 350 RPM, accompanied by the addition of 1% ethanol to promote subsequent phase separation. In addition, it was centrifuged at 1000 RPM to separate the solvent from the soap.
9. Two extractions and two more centrifugations were carried out as previously described above.
10. The solvent of the dispersing phases of each of the centrifugations contemplated in the previous steps was evaporated and recovered by means of a battery of evaporators arranged in series, which are operated under pressures from 10 to 80 Pa and at temperatures between 40 to 90° C., where after evaporation, the unsaponifiable matter (MI) that was dissolved in the solvent was obtained;
11. The soap obtained from the last centrifugation of the tenth step, that is, the most spent soap, was arranged for acidification with an acid selected from citric acid in an aqueous solution at 67% w/w in an ultrasonic reactor, where the ratio of soap to the acid solution was 2:1 by weight and at a temperature of 75° C. for 2 hours with constant stirring. Then, it was cooled using water from a tower and the water was then separated from the free fatty acids formed using a centrifuge that operated at 650 RPM.
12. The esterification of the free fatty acids was carried out in an ultrasonic reactor using ethanol, feeding 1 part of activated ethanol for every 4 parts of free fatty acids. The ethanol activation was carried out by dissolving 7.5% sulfuric acid in the alcohol. The reaction was carried out for 1.5 hour at a temperature of 70° C. with stirring and constant reflux of ethanol. Finally, a stream of fatty acids was obtained in the form of the ethyl ester.
13. Additionally, a wash was carried out on the previously obtained unsaponifiable matter, using a 10% aqueous solution of calcium chloride. The washing was carried out in a ratio of 1:3 by weight with respect to the desolventized unsaponifiable matter, and the washing temperature was 45° C.
14. The separation of the washing solution was carried out by centrifugation, this stage was carried out at 2000 RPM.
15. 12 parts of acetone were added for each mass part of unsaponifiable matter, it was heated to 30° C. and stirred until dissolved. Then it was crystallized between 5° C. and −10° C., and further maintained under isothermal conditions at 5° C. for 8 hours, then cooling to −10° C. and then further maintained under isothermal conditions at this temperature for 10 hours.
16. It was then centrifuged and filtered using a basket centrifuge to obtain cholesterol crystals with a purity of 78.6%.
17. 12 parts of acetone were then added to each part of cholesterol with a purity greater than 70%, the mixture was heated to 30° C. and stirred until dissolved, where it then crystallized at a temperature between −40° C. and 0° C., while maintaining isothermal conditions at −5° C. for 10 hours, then cooling to −40° C. and further maintaining isothermal conditions at this temperature for 8 hours.
18. Finally, the separation of the crystals obtained from the solvent is carried out using a basket centrifuge in order to obtain cholesterol crystals with a purity of 97.8%.

### Example 6

1. 2000 kg of fish oil with an acid value of 1.37 mg KOH/g were transesterified with 600 kg of ethanol and 15 kg of sodium ethoxide in four ultrasonic reactors arranged in series within a period of 50 minutes, at a pressure of 3 bar, at a temperature of 65° C. and constant reflux of alcohol.
2. The ethanol that was not consumed by the reaction was recovered in an evaporator that operates at a pressure of 0.5 bar and 60° C.
3. The ethyl ester and glycerin mixture was separated using a disc centrifuge with a spin speed of 4500 RPM. Once the glycerin was removed, the analysis of partial glycerides and ethyl ester confirmed that the ethyl ester concentration was 96.3%;
4. Eight short-path stills were arranged in series, where the residue from one still is fed to the next still and the distillate is returned to the previous still, at temperature ranging between 125 and 160° C. and vacuum pressures from 0.1 to 7 Pa. At the end of this stage, two streams were obtained: a stream with a high cholesterol content and another stream with a high content of EPA and DHA fatty acids.
5. 100 kg of the stream with a high concentration of cholesterol were obtained, which stream was saponified using 100 kg of an aqueous solution of sodium hydroxide at 15% w/w. The reaction time was two hours at a temperature of 75° C., stirring at 750 RPM and with constant reflux.
6. The obtained soap then enters an extractor column in which a liquid-liquid extraction is carried out using toluene in a ratio of 2 to 1 by weight with respect to the soap being fed. The extraction is carried out with constant stirring at 400 RPM and at a temperature of 40° C.
7. The extraction mixture is then centrifuged for the separation of soap and solvent in a disc centrifuge at 1000 RPM.
8. A new extraction of the previously extracted soap was carried out in another extracting column using diethyl ether in a ratio of 2 to 1 with respect to the soap being fed, with constant stirring at 350 RPM, accompanied by the addition of 1% ethanol to favor subsequent phase separation. In addition, it was centrifuged at 1000 RPM to separate the solvent from the soap.
9. Three extractions and three more centrifugations were carried out as previously described above.
10. The solvent from the dispersing phases of each of the centrifugations conducted in the previous steps was evaporated and recovered by means of a battery of evaporators arranged in series, which operated under pressures from 10 to 80 Pa and temperatures between 40 and 90° C., wherein after evaporation, the unsaponifiable matter (MI) that was dissolved in the solvent was obtained.
11. The soap obtained from the last centrifugation of the tenth step, that is, the most spent soap, was arranged for acidification with an acid selected from citric acid in an aqueous solution at 67% w/w in an ultrasonic reactor, where the ratio of soap to the acid solution was 2:1 by weight and at a temperature of 75° C. for 2 hours with constant stirring. Then, it was cooled using water from a tower and the water was then separated from the free fatty acids formed using a centrifuge that operated at 650 RPM.
12. The esterification of the free fatty acids was carried out in an ultrasonic reactor using ethanol, feeding 1 part of activated ethanol for every 4 parts of free fatty acids. Ethanol activation was performed by dissolving 7.5% p-toluenesulfonic acid in alcohol. The reaction was carried out for 1.5 hour at a temperature of 70° C. with stirring and constant reflux of ethanol. Finally, a stream of fatty acids was obtained in the form of the ethyl ester.
13. Additionally, a wash was carried out on the previously obtained unsaponifiable matter, using a 10% aqueous potassium hydroxide solution. The washing was carried out in a ratio of 1:3 by weight with respect to the desolventized unsaponifiable matter, at a washing temperature of 45° C.
14. The separation of the washing solution was carried out by centrifugation, and it was carried out at 2000 RPM.
15. 12 parts of benzene were added for each part of unsaponifiable matter, it was then heated to 30° C. and stirred until dissolved. Then it was crystallized between 5° C. and −10° C., and maintained under isothermal conditions at 5° C. for 8 hours, then cooling to −10° C. and further maintained under isothermal conditions at this temperature for 10 hours.
16. It was then centrifuged and filtered using a basket centrifuge to obtain cholesterol crystals with a purity of 73.4%.
17. 12 parts of diethyl ether were added to each part of cholesterol with a purity greater than 70%, the mixture was then heated to 30° C. and stirred until dissolved, where it then crystallized at a temperature between −40° C. and 0° C., and then maintained under isothermal conditions at −5° C. for 10 hours, then cooling to −40° C. and further maintaining under isothermal conditions at this temperature for 8 hours.
18. Finally, the separation of the crystals obtained from the solvent is carried out using a basket centrifuge in order to obtain cholesterol crystals with a purity of 92.7%.

Therefore, an object of the present invention is to provide a process for producing cholesterol from fish oil, generally comprising the steps of transesterifying the refined oil using a basic catalyst. In the next step, there is obtained a stream concentrated in cholesterol and another stream concentrated in omega-3 fatty acids said fatty acids being EPA and DHA through molecular distillation. In the subsequent step, saponification of the cholesterol-rich stream is conducted using bases such as potassium hydroxide, sodium hydroxide, or calcium chloride. The mixture is saponified for a time and temperature with constant stirring and reflux. Several extractions with solvents such as toluene, ethylene dichloride, methylene chloride, diethyl ether, petroleum ether, acetone, benzene or hexane and centrifugations are also carried out to obtain the unsaponifiable matter. The last stream is crystallized using acetone, benzene, toluene, diethyl ether, petroleum ether or hexane to obtain cholesterol with a minimum purity of 95%. In addition, the soap obtained from saponification is converted to the ethyl ester forms by dual reactions that is acidification of the soap and then acid esterification.

Only some preferred embodiments of the invention have been illustrated by way of example. In this regard, the cholesterol separation process present in the heavy fraction from fish oil will be appreciated, as well as the configurative arrangements that can be chosen from a plurality of alternatives without departing from the spirit of the invention according to the following claims.

1-8. (canceled)

9. A process for separating cholesterol from the heavy fraction of fish oil, said process comprising a first stage of obtaining fatty acids in the form of their esters and wherein said first stage comprises the following steps:
(i) producing a mixture of fatty acid esters and glycerin by transesterifying refined fish oil with an acid value of less than 2 mg KOH/g by reacting with an alcohol selected from the from the group consisting of methanol, propanol, butanol or ethanol in a ratio between 1:5 and 1:2 w/w with respect to the oil refined, preferably 1:3 w/w in the presence of a basic catalyst selected from the group consisting of sodium hydroxide, potassium hydroxide, sodium ethoxide or sodium methoxide in a concentration between 0.75 and 1.5% w/w with respect to the oil, said reaction conducted in four ultrasonic reactors arranged in series for a time of between 20 and 60 minutes, at a pressure between 1 and 3 bar, at a temperature between 60° C. and 90° C. and a constant reflux of alcohol;
(ii) recovering the alcohol not consumed in the reaction by flash evaporation in an evaporator that operates between 0.2 and 1 bar of pressure and at a temperature between 50 and 80° C., wherein the alcohol recovered in this step is reusable in the first step;
(iii) separating the mixture of the fatty acid esters and glycerin obtained from the transesterification by means of a disc centrifuge that operates between 3500 and 6000 RPM, wherein said reaction of step (i) achieves a conversion of glycerides to esters of at least 95%;
(iv) concentrating the cholesterol present in the oil in the esterified form using molecular distillation, using short-path distillation units arranged in series, where the residue from one evaporator is fed to the next distillation unit and the distillate is returned to the previous still, where the operating conditions of the stills comprise vacuum pressures from 0.1 to 10 Pa, temperatures between 120° C. and 165° C. and heat transfer areas between 2 m² and 5 m² and wherein a stream with high cholesterol content and another stream of esters with high content of EPA and DHA fatty acids is obtained.

10. The process according to claim 9, further comprising a second stage of obtaining saponification and extraction products which second stage comprises the following steps:
(v) making a soap by reacting in a high efficiency mixer the concentrated fraction of cholesterol in weight ratio between 1:0.9; or 1:1.1; or 1:1.2, preferably 1:1 with respect to the stream coming from molecular distillation with a base selected from the group consisting of calcium hydroxide, sodium hydroxide, potassium hydroxide or calcium chloride in water at a concentration between 10% and 30% w/w, with the addition of an alcohol selected from the group consisting of ethanol, methanol, glycerin or propylene glycol with a ratio between 2 to 5% w/w, preferably 3% w/w with respect to the base solution and wherein said reaction mixture is saponified for a time between 1 and 4 hours, preferably 1 to 2 hours, at a temperature between 60° C. and 85° C. with stirring between 500 to 1050 RPM using with constant reflux;
(vi) extracting the soap from the fifth step using an extracting column by adding an extracting solvent selected from the group consisting of toluene, ethylene dichloride, methylene chloride with a ratio of 4:1 to 1:1, preferably 3:1 with respect to the soap being fed, with constant stirring between 200 RPM and 750 RPM, and at a temperature between 30-50° C. thereby forming two phases;
(vii) optionally separating the phases by the addition of an alcohol selected from the group consisting of ethanol, methanol, propanol or butanol in a concentration of 0 to 5% by weight with respect to the mixture that includes the extraction solvent and the soap;
(viii) centrifuging the extraction mixture for the separation of soap and solvent at a speed range between 500 and 3500 rpm;
(ix) further extracting the centrifuged soap resulting from the eighth step in an extracting column, by adding a solvent selected from the group consisting of diethyl ether, petroleum ether, acetone, benzene or hexane at a ratio of 4:1 to 1:1, with respect to the soap being fed, using constant agitation and at a temperature between 30 to 55° C.;
(x) performing steps six through ninth 1 to 3 more times;
(xi) recovering by means of evaporation the solvents used in the eighth, ninth and tenth steps using a battery of evaporators arranged in series, which operate under pressures from 10 to 100 Pa and temperatures between 30 to 120° C., wherein after evaporation, the unsaponifiable matter (MI) that was dissolved in the solvent is obtained;
(xii) acidifying the soap obtained from the tenth step with an acid selected from the group consisting of sulfuric acid, hydrochloric acid, acetic acid, phosphoric acid or citric acid in an aqueous solution at 50 to 70% w/w in an ultrasonic reactor, where the ratio of soap to acid solution is between 3:1 to 1:1, preferably 2:1 and a temperature between 70° C. and 90° C. for 1 to 3 hours with constant stirring, then cooled and separating the water from the free fatty acids formed using a centrifuge that operates in a range between 450 and 850 RPM;
(xiii) esterifying the free fatty acids in an ultrasonic reactor using an alcohol selected from the group consisting of ethanol, methanol or propanol in ratios to the free fatty acid by weight between 1:5 to 1:3, preferably 1:4 and an acid catalyst selected from the group consisting of sulfuric acid, p-toluenesulfonic acid, citric acid, acetic acid, phosphoric acid or hydrochloric acid dissolved in the alcohol at a concentration of 3 to 8% w/w for one to three hours at a temperature between 65° C. and 85° C. with stirring and constant reflux thereby obtaining a stream of fatty acids in the form of their esters.

11. The process according to claim 10, further comprising a third stage of cholesterol purification and crystallization which third stage comprises the following steps:
(xiv) washing the unsaponifiable matter with an aqueous solution of sodium hydroxide or calcium chloride at 5-10%; wherein the washing is carried out in a proportion of 1:2 and 1:4 by weight with respect to the desolventized unsaponifiable matter at a temperature range between and 60° C.;
(xv) centrifuging of the mixture obtained after washing to separate the unsaponifiable matter treated and the washing water, which step is conducted at a range of speeds between 500 and 3500 RPM, preferably 2000 RPM;
(xvi) adding an organic solvent selected from the group consisting of acetone, benzene or toluene to the unsaponifiable material from the fifteenth step at a ratio of 15:1 to 5:1, then heated to 30° C. and stirred until dissolved and then crystallized at a temperature between −10° C. and 5° C. and maintained under isothermal conditions at 5° C. for 8 hours, then cooling to −10° C. for 10 hours;
(xvii) centrifuging and filtering the resulting product from the sixteenth step using a basket centrifuge to obtain cholesterol crystals with a purity greater than 70%;
(xviii) adding another organic solvent selected from the group consisting of diethyl ether, petroleum ether or hexane to the greater than 70% purity cholesterol from the seventeenth step at a ratio of 15:1 to 5:1, heated to 30° C. and stirred until dissolution, and then crystallized at a temperature between −40° C. and 0° C., and then maintained under isothermal conditions at −5° C. for 10 hours, and then cooled to −40° C. for 8 hours; and
(xix) centrifuging and filtering the product from the eighteenth step using a basket centrifuge wherein cholesterol crystals with a purity greater than 95% are obtained.

12. The process according to claim 9, wherein said alcohol is ethanol.

13. The process according to claim 9, wherein said alcohol is methanol.

14. The process according to claim 9, wherein said basic catalyst is potassium hydroxide.

15. The process according to claim 9, wherein said basic catalyst is sodium ethoxide.

16. The process according to claim 9, wherein said basic catalyst is sodium methoxide.

17. The process according to claim 9, wherein said basic catalyst is sodium hydroxide.

18. The process according to claim 10, wherein said base in step (v) is potassium hydroxide.

19. The process according to claim 10, wherein said base in step (v) is calcium hydroxide.

20. The process according to claim 10, wherein said extracting solvent in step (vi) is ethylene dichloride.

21. The process according to claim 10, wherein said acid used in step (xii) is sulfuric acid.

22. The process according to claim 10, wherein said acid used in step (xii) is hydrochloric acid.

23. The process according to claim 10, wherein said alcohol used in step (xiii) is ethyl alcohol.

24. The process according to claim 10, wherein said catalyst used in step (xiii) is p-toluenesulfonic acid.

25. The process according to claim 11, wherein said aqueous solution used in step (xiv) is sodium hydroxide.

26. The process according to claim 11, wherein the organic solvent used in step (xvi) is acetone.

27. The process according to claim 11, wherein the organic solvent used in step (xviii) is diethyl ether.

28. The process according to claim 11, wherein the organic solvent used in step (xviii) is petroleum ether.
